(19) United States
(12) Patent Application Publication
(10) Pub. No.: US 2021/0299051 A1
(43) Pub. Date: Sep. 30, 2021

(54) PHARMACEUTICAL COMPOSITION FOR AMELIORATION OF ADVERSE SIDE EFFECTS

(71) Applicant: Samuel Messinger, Ramot Bet Shemish (IL)

(72) Inventor: Samuel Messinger, Ramot Bet Shemish (IL)

(21) Appl. No.: 16/833,525

(22) Filed: Mar. 28, 2020

(51) Int. Cl. A61K 9/24; A61K 9/48; A61K 9/20

(52) U.S. Cl. CPC A61K 9/209; A61K 9/4808; A61K 45/06; A61K 9/2054; A61K 9/2027

(57) ABSTRACT

The present invention provides a pharmaceutical composition (100) with one or more layers comprising a drug(s) to treat a disease, and the remaining layer(s) comprise ingredients to block adverse side effects of the drug(s). One pill comprises: a first outer layer (102) formed with a primary medication (106), a second inner layer (104) formed with a secondary medication (108), and a film coating adapted to cover or be part of the second layer (104). The first layer (102) allows immediate release of the primary medication (106) and the second layer (104) allows sustained release of the secondary medication (108). Further, the film coating allows delayed release of the second layer (104) and medication (108). Another embodiment comprises a pharmaceutical composition comprising three layers with three different medications: a first outer layer (202), a second middle layer (204), a third inner layer (206), and two layers of film coating.

### FIELD OF THE INVENTION

The present invention generally relates to pharmaceutical and chemical tablets or pills. More particularly, the present invention relates to a pharmaceutical composition with one or more layers of a drug (an active ingredient) and additives (inactive ingredients), each to be released into a user's bloodstream at a certain time period, thereby reducing the adverse side effects during treatment, e.g. such as those associated with taking the active ingredient

### BACKGROUND OF THE INVENTION

A medication or pill is an actual dosage form of a tablet, a capsule, suppository, transdermal patch, or solution. The medication consists of a drug (active ingredient) and additives (inactive ingredients). The active ingredient (drug) is a chemical substance that is taken to produce a desired effect, such as lowering blood pressure. The additives (inactive ingredients), such as diluents, stabilizers, disintegrates, and lubricants, are mixed with the drug to make it easier to swallow or to help break it up in the gastrointestinal tract. For example, to prepare tablets, the active/inactive ingredient mixture may be formed into small grains and compressed into a tablet form. The type and amount of the additives and the degree of compression affect how quickly the tablet disintegrates and absorbed. Drug manufacturers adjust these variables to optimize absorption.

Generally, any medication or pill could have an adverse effect, whether a prescription drug, an over the counter (OTC) drug, an alternative, herbal or complementary therapy, or a vitamin supplement. Adverse side effects happen when the medication or pill or drug causes a problem because the pill or the drug does more than to treat a target tissue or medical condition. The impact could range from minor to severe and life-threatening problems, which includes, but is not limited to headache, insomnia, nausea, dizziness, drowsiness, diarrhea, abnormal heart rhythms, and cancer.

It is important to note that the side effects from the medication or pill could vary widely, from mild nausea to death. Different pills have different side effects. Some pills could not help but trigger adverse side effects because of their chemical structure.

Lf a tablet releases the drug too quickly, the blood level may become too high, causing an excessive response. If the tablet does not release the drug quickly enough, much of the drug may be eliminated in the feces without being absorbed, and the blood level may be too tow. For example, Vicodin™ is a popular drug for treating acute or chronic, moderate to advanced severe pain. Its most common side effects are light-headedness, dizziness, sedation, nausea, and vomiting. Vicodin could reduce breathing, impair thinking, limit physical abilities, and be habit forming.

In another example, Simvastatin™ is one of the first “statins” (HMG-CoA reductase inhibitors) and is approved for treating high cholesterol and reducing the risk of stroke, death from heart diseases, and risk of heart attacks. The most common side effects of Simvastatin are headache, nausea, vomiting, diarrhea, abdominal pain, and muscle pain. Like other statins, Simvastatin can cause muscle break down.

In yet another example, Lisinopril™ is described which is an angiotensin converting enzyme (ACE) inhibitor, and is used for treating high blood pressure, congestive heart failure, and for preventing kidney failure caused by high blood pressure and diabetes. Lisinopril side effects include dizziness, nausea, headaches, drowsiness, and sexual dysfunction. ACE inhibitors may cause a dry cough that resolves when the drug is discontinued.

In yet another example, Levothyroxine™ is described which is a man-made version of the thyroid hormone. Levothyroxine is used for treating hypothyroidism and its side effects usually result from high levels of thyroid hormone. Excessive thyroid hormone can cause chest pain, increased heart rate, excessive sweating, heat intolerance, nervousness, headache, and weight loss.

In yet another example, Azithromycin™ is an antibiotic that is used for treating ear, throat, and sinus infections, as well as pneumonia, bronchitis, and some sexually transmitted diseases. The common side effects of Azithromycin include loose stools, nausea, stomach pain, and vomiting. Rare side effects include abnormal liver tests, allergic reactions, nervousness, and abnormal heart beats.

In yet another example, Metformin™ is used alone, or in combination with other drugs, for treating type 2 diabetes in adults and children. The most common side effects of Metformin are nausea, vomiting, gas, bloating, diarrhea, and loss of appetite.

In yet another example, Lipitor™ is a “statin” (HMG-CoA reductase inhibitors) and is approved for treating high cholesterol. Lipitor also prevents chest pain, stroke, and heart attack in individuals with coronary artery disease. Lipitor causes minor side effects, such as constipation, diarrhea, fatigue, gas, heartburn, and headache. Like other statins, Lipitor can cause muscle pain and muscle break down.

In yet another example, Amlodipine™ is a calcium channel blocker used for treating high blood pressure and for the treatment and prevention of chest pain. The most common side effects of Amlodipine are headache and swelling of the lower extremities. Amlodipine can also cause dizziness, flushing, fatigue, nausea, and palpitations.

In yet another example, Amoxicillin™ is a penicillin type antibiotic used for treating several types of bacterial infections, such as: ear, tonsils, throat, larynx, urinary tract, and skin infections. The side effects of Amoxicillin are diarrhea, heartburn, nausea, itching, vomiting, confusion, abdominal pain, rash, and allergic reactions.

Further yet another example includes Hydrochlorothiazide™, which is a diuretic (water pill) that is used alone or combined with other drugs for treating high blood pressure. The side effects of Hydrochlorothiazide include weakness, low blood pressure, light sensitivity, impotence, nausea, abdominal pain, electrolyte disturbances, and rash.

Further yet another example includes steroid drugs, such as hydrocortisone. The adverse side effects are extensive, such as: impaired immunity; impaired glucose tolerance; gastrointestinal tract ulcers and perforation; mood alterations; etc.

Therefore, many drugs currently being used to treat diseases are often ineffective in part due to the lack of absorption into targeted tissues (e.g. bloodstream), and could ultimately demand the use of additional dose, or alternative types of medications, each potentially producing one or more undesirable adverse side effects. Accordingly, there is a need for a pharmaceutical composition (e.g. in a tablet, pill, capsule, etc. form) with one or more layers of a drug (an active ingredient of main drug) and additives (inactive ingredients or ingredients to treat adverse side effects of main drug) each to be released into the bloodstream at a certain time period, thereby effectively reducing adverse side effects during treatment while increasing targeted drug delivery.

### SUMMARY OF THE INVENTION

The following presents a simplified summary of the invention to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

It is, therefore, an object of the present invention to provide a pharmaceutical composition that aids in ameliorating a disease, as well as reducing the adverse side effects associated with administering the pharmaceutical composition.

It is another object of the present invention to provide a pharmaceutical composition which comprises more than one layer of a drug or active ingredient and additives, each layer comprising a different drug.

It is another object of the present invention to provide a pharmaceutical composition which comprises more than one layer of immediate release or sustained release drug or active ingredient or a combination thereof.

It is still another object of the present invention to provide a pharmaceutical composition which releases the drug or active ingredient in the bloodstream when the drug reaches the lower gastrointestinal tract within a user's body.

In another embodiment, the pill is designed with one or more layers of a drug or medication (an active ingredient) and additives (inactive ingredients) each to be coated with a specific coating according to a desired release.

Accordingly, in an aspect, the present invention provides a pharmaceutical composition comprising a first layer, a second layer and a film coating. The first layer is formed with a primary medication, the second layer is formed with a secondary medication and the film coating is adapted to cover the second layer. The first layer allows immediate release of the first primary medication and the second layer allows sustained release of the second or secondary medication. Further, the film coating prevents the second layer from dissolving until the second layer reaches lower gastrointestinal tract within a user's body, thereby releasing the secondary medication into bloodstream after a period of delay after oral ingestion.

Either the primary or the secondary medication comprises the active ingredients to treat the disease, and other drug layer provides the ingredients to reduce the adverse side effects of the active ingredients. For example, in a first embodiment, the primary medication is the outer layer with the active drug ingredients to treat cancer and is immediately released in the upper gastrointestinal (GI) tract. Then the secondary medication comprises ingredients that block the adverse effects of the cancer drug and is released in the lower GI tract.

Accordingly, in another aspect, the present invention provides a pharmaceutical composition comprising a first layer, a second layer, a third layer, and a plurality of film coatings. The first layer is formed with a first (e.g. primary) medication, the second layer is formed with a secondary medication, and the third layer is formed with a tertiary medication. Any one or two of the medications may comprise active ingredients to treat a disease/disorder, and the remaining one or two drugs may comprise ingredients to reduce the adverse side effects associated with the active ingredients.

The plurality of film coatings is adapted to cover the second layer and the third layer (e.g. two film coatings total). In one embodiment, the first drug and the second drug are immediately released respectively, in the upper GI tract, while the third layer allows sustained release of the tertiary medication in the lower GI tract.

In another embodiment, the first drug is immediately released in the upper GI tract; and then the second and third drug are released in the middle and/or lower GI tract. For example, the plurality of film coatings prevent the second layer and the third layer from dissolving until the second layer and the third layer reach the lower gastrointestinal tract within a user's body, thereby releasing the secondary medication and the tertiary medication into a patient's bloodstream after a period of delay after oral ingestion.

The pharmaceutical composition in the present invention overcomes the foregoing disadvantages inherent in the prior art; and the general purpose is to provide a pharmaceutical composition that is capable of including all advantages of the prior art while also overcoming the drawbacks inherent in the prior art.

Other aspects, advantages, and salient features of the invention will become apparent to those skilled in the art from the following detailed description, which, details the invention in different embodiments.

### BRIEF DESCRIPTION OF THE DRAWINGS

The embodiments herein will be better understood from the following detailed description with reference to the drawings, in which:

FIG. 1 illustrates a cross-sectional view of a pharmaceutical composition in a tablet form of a first embodiment comprising: a first outer layer for immediate release of the primary medication and the second inner layer allows sustained release of the secondary medication, wherein the primary and secondary medications comprise different or the same drug;

FIG. 2 illustrates a sectional view of the pharmaceutical composition in a tablet form of FIG. 1 illustrating a cut-out of the first outer layer covering an intact second inner layer.

FIG. 3 illustrates a sectional view of a second exemplary pharmaceutical composition in accordance with another embodiment of the present invention, comprising a first outer layer formed with a first (e.g. primary) medication, a second middle layer formed with a second (e.g. secondary) medication, a third inner layer formed with a tertiary medication; and a plurality of film coatings adapted to cover said second layer and said third layer to delay release of the secondary and tertiary medication; and

FIG. 4 is a flowchart of steps in the method of manufacturing the pharmaceutical composition of the first embodiment.

### DETAILED DESCRIPTION OF THE INVENTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The use of terms “including,” “comprising,” or “having” and variations thereof herein are meant to encompass the items listed thereafter and equivalents thereof as well as additional items. Further, the terms, “an” and “a” herein do not denote a limitation of quantity, but rather denote the presence of at least one of the referenced items.

As used herein, the term “primary medication” refers to the first drug to be released in the patient's body and is in the outermost layer of the pill.

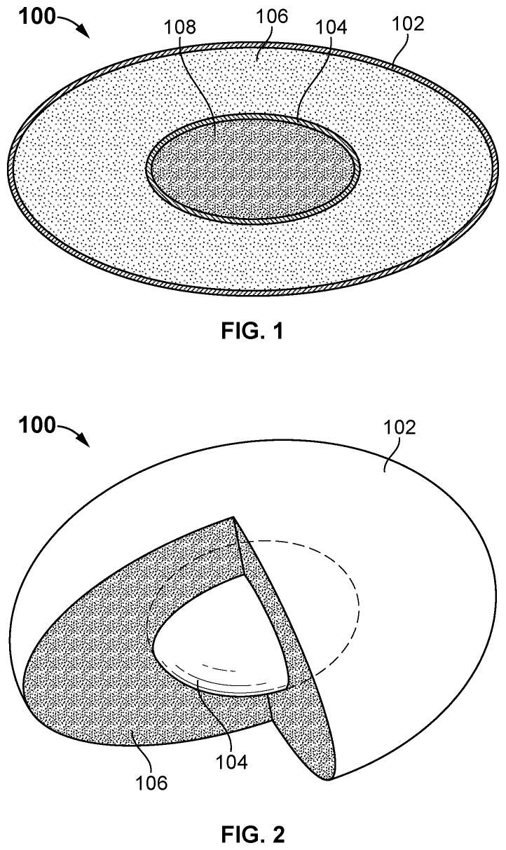

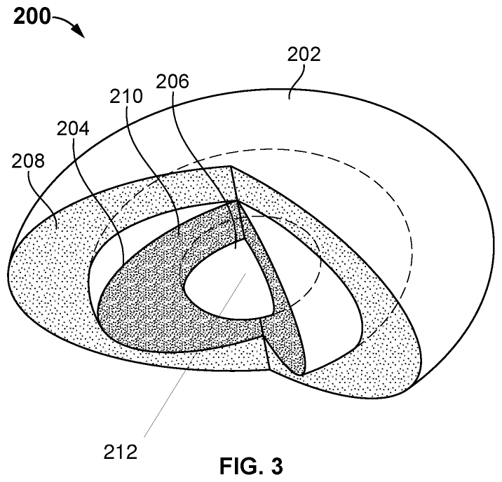

As used herein, the term “secondary medication” refers to the second inner (FIG. 1, 2) drug or second middle (FIG. 3) drug to be released in the patient's body, and is the layer beneath the primary medication and the first film coating.

As used herein, the term “tertiary medication” refers to the third inner drug (FIG. 3) to be release in the patient's body, and it is beneath the second film coating.

As used herein, the term “amelioration formula” refers to the layer(s) comprising an ingredients or additives that counteracts the adverse side effects of the main drug that is administered to treat or prevent a disease or disorder.

It is noted that the figures discussed herein are for exemplary embodiments comprising: two layers of different drugs (FIGS. 1, 2, 4) and three layers of different drugs (FIG. 3). In an embodiment of FIGS. 1, 2 and 4, the primary medication comprises the active ingredients for treating the disease/disorder, while the remaining layers (e.g. secondary and/or tertiary medication) comprise ingredients for treating adverse side effects of the primary medication.

In another embodiment of FIGS. 1, 2, and 4, the primary medication comprises ingredients to treat the adverse side effects associated with the active ingredients in the secondary medication.

Referring to the drawings, the invention will now be described in more detail.

A pharmaceutical composition (100), as shown in FIG. 1, comprises a first outer layer (102) and a second inner layer (104). The pharmaceutical composition (100) refers to a dosage form suitable for oral administration, such as tablets, granules, powder, spheroids, pellets, pills, capsule and the like. Preferably, the pharmaceutical composition (100) is a tablet, a pill or a capsule. Further, the tablets or pills may be, but not limited to, buccal, sub-lingual, or orally disintegrating dosage forms.

In accordance with an exemplary embodiment of the present invention, the first outer layer (102) is formed with a first (e.g. primary) medication (106). The first outer layer (102) allows immediate release of the primary medication (106). The primary medication (106) is a plurality of additives for preventing adverse side effects during treatment. The first outer layer (102) facilitates instant disintegration of the primary medication (106) after administration. Further, the primary medication (106) comprises, but is not limited to: domperidone, procyclidine, ranitidine, Co enzyme-Q10, vitamin D, and mebeverine. Preferably the primary medication (106) comprises domperidone mixed with lactose monohydrate or maize starch.

In accordance with the exemplary embodiment of the present invention, the second inner layer (104) is formed with a secondary medication (108). The second inner layer (104) allows sustained release of the secondary medication (108). The secondary medication (108) is an active agent or active ingredient. In other words, the second inner layer (104) facilitates delivery of a certain amount of the secondary medication (108) for a prolonged period of time to a targeted diseased area within the body.

In accordance with an embodiment of the present invention, the secondary medication (108) comprises a drug selected from the group consisting of, but not limited to, azithromycin, haloperidol, co-amoxiclav, metformin, rosuvastatin, levofloxacin, moxifloxacin, erythromycin, clarithromycin, and ofloxacin. Preferably, the secondary medication (108) comprises azithromycin mixed with dibasic calcium phosphate anhydrous, pregelatinized starch, sodium croscarmellose, magnesium stearate, sodium lauryl sulfate, hypromellose, lactose, titanium dioxide or triacetin.

In accordance with an embodiment of the present invention, a film coating is adapted to cover the second inner layer (104). The film coating prevents the second inner layer (104) from dissolving until the second inner layer (104) reaches the lower gastrointestinal tract within the user's body, thereby releasing the secondary medication (108) into the bloodstream after a period of delay after oral ingestion or oral administration.

In accordance with an embodiment of the present invention, the film coating is an enteric coating made up of acid-resistant polymers. Further, the film coating is made up of a material selected from the group consisting of, but not limited to, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate, trimellitate, sodium alginate, zein or enteric coating aqueous solution (ethylcellulose, medium chain triglycerides, oleic acid, sodium alginate, stearic acid). Preferably, the film coating is made up of cellulose acetate succinate.

In accordance with an embodiment of the present invention, the film coating is an enteric coating made up of acid-resistant polymers and/or pH-sensitive polymeric coats to delay the release of the secondary medication (108) into the bloodstream within the user's body. The pH-sensitive polymeric coats are configured to protect the second layer (104) of the composition (100) from the acidic environment within the user's stomach. Further, the acid-resistant polymers include, but is not limited to, the Eudragit acrylic polymers (e.g. Nikam et al., Pharmacologyonline 1: 152-164 (2011) the entire contents of which are incorporated by reference).

In accordance with an exemplary embodiment of the present invention, the pharmaceutical composition (100) is designed using two different medications with multi-layer coatings and also applied in various dosage forms. For example, the first outer layer (102) of the composition (100) is dissolved in the small intestine at a pH more than 5.5, and the second inner layer (104) is dissolved in the colon at a pH more than 7 within the user's body. This is achieved by using either layer coating or by preparation of two different coated granules. Both the primary medication (106) and the secondary medication (108) can be designed to absorbed in specific sections of the digestive system, from the stomach through the colon.

In another example, the primary medication (106) is released within, but not limited to, one to two hours, after oral administration and provides day-time treatment for gastroesophageal reflux disease (GORD); and the secondary medication (108) is released at about, but not limited to, five to six hours after oral administration and addresses breakthrough GORD, which is often seen in late evening or overnight. In a further example, disintegrates could also be added to the second inner layer (104) of the composition, which is covered with Eudragit S layer in order to facilitate targeted release of the secondary medication (108).

In accordance with an embodiment of the present invention, the second inner layer (104) of the composition (100) is formed of the secondary medication (108), which is an active agent in the core of the composition (100). Further, the first outer layer (102) formed of the primary medication (106) includes the additives or drugs which counter the adverse side effects of the active agent during the treatment.

In accordance with an embodiment of the present invention, the secondary medication (108) may be released in other parts of the body than the primary medication (106), such as a targeted drug delivery to a specific diseased area within the body.

In accordance with an embodiment of the present invention, the pharmaceutical composition (100) is designed with one or more layers of a drug or medication (an active ingredient) and additives (inactive ingredients), wherein each of the layers is coated with a specific coating according to a desired release of the drug and additives.

In yet another embodiment, both the cure and side effect treatment can be delivered by means of cream, gel, liniment, balm, lotion, or ointment.

In another embodiment, both the cure and side effect treatment can be delivered as ear drops (otic), eye drops (ophthalmic), skin patch (transdermal), vaginal rings, dermal patch, or externally applied powder.

In yet another embodiment, both the cure and side effect treatment can be delivered by means of vaginal douche or pessary, or rectal, urethral, or nasal suppository.

In accordance with an embodiment of the present invention, the pharmaceutical composition for both the cure and the adverse side effect treatment is adapted to be delivered intradermally, subcutaneously, intramuscularly, intraosseously, intraperitoneally, or intravenously.

In yet another embodiment, both the cure and side effect treatment can be delivered by means of cream, gel, liniment, balm, lotion, or ointment.

In another embodiment, both the cure and side effect treatment can be delivered as ear drops (otic), eye drops (ophthalmic), skin patch (transdermal), vaginal rings, dermal patch, or externally applied powder.

In yet another embodiment, both the cure and side effect treatment can be delivered by means of vaginal douche or pessary, or rectal, urethral, or nasal suppository,

### Three Layered Tablet

In another embodiment, the pill is designed with one or more layers of a drug or medication (an active ingredient) and additives (inactive ingredients) each to be released into the user's bloodstream by a certain time period, thereby avoiding side effects during treatment. In one embodiment, the pill comprises at least, but not limited to, a first layer, a second layer, and a third layer or more. In one embodiment, the first layer is formed with a first primary medication, the second layer is formed with a secondary medication, and the third medication is formed with a tertiary medication and same for all other layers.

In an exemplary embodiment, the primary medication comprises an active agent or ingredients to treat a disease/disorder, and the second medication and the third medication comprise additives for reducing and/or preventing the adverse side effects associated with the primary medication during treatment.

In another exemplary embodiment, the primary and secondary medications comprise additives for reducing and/or preventing the adverse side effects associated with the tertiary medication that comprises active ingredients for treating a disease/disorder.

In another exemplary embodiment, the primary and tertiary medications comprise additives for reducing and/or preventing the adverse side effects associated with the secondary medication that comprises active ingredients for treating a disease/disorder.

In another exemplary embodiment, the primary medication comprises additives for reducing and/or preventing the adverse side effects associated with the secondary and/or tertiary medication that comprise active ingredients for treating one or more diseases/disorders.

In another exemplary embodiment, the tertiary medication comprises additives for reducing and/or preventing the adverse side effects associated with the primary and the secondary medication that comprise active ingredients for treating one or more diseases/disorders.

In another exemplary embodiment, the secondary medication comprises additives for reducing and/or preventing the adverse side effects associated with the primary and the tertiary medication that comprise active ingredients for treating one or more diseases/disorders.

For example, in accordance with a second embodiment of the present invention, a pharmaceutical composition (200), as shown in FIG. 3, is disclosed. The pharmaceutical composition (200) comprises a first outer layer (202), a second middle layer (204) and a third inner layer (206). The pharmaceutical composition (200) refers to a dosage form suitable for oral administration, such as tablets, granules, powder, spheroids, pellets, pills, capsule and the like. Preferably, the pharmaceutical composition (200) is a tablet, a pill or a capsule. Further, the tablets or pills may be, but not limited to, buccal, sub-lingual, or orally disintegrating dosage forms.

In accordance with an embodiment of the present invention, the first outer layer (202) is formed with a primary medication (208), and the second middle layer (204) is formed with a secondary medication (210). Each of the first outer layer (202) and the second middle layer (204) allows immediate release of the primary medication (208) and the secondary medication (210), respectively. Each of the primary medication (208) and the secondary mediation (210) comprises a plurality of additives for preventing adverse side effects during treatment, such as from the third inner layer (206), as wells as the layers (202) and (204). Further, each of the first outer layer (202) and second middle layer (204) facilitates instant disintegration of the primary medication (208) and secondary medication (210), respectively, after administration. Furthermore, each of the primary medication (208) and the secondary medication (210) comprises, but are not limited to, domperidone, procyclidine, ranitidine, co enzyme-Q10, vitamin D, and mebeverine.

In accordance with an embodiment of the present invention, the third inner layer (206) is formed with a tertiary medication (212) The third inner layer (206) allows sustained release of the tertiary medication. The tertiary medication is an active agent or active ingredient. In other words, the third inner layer (206) facilitates delivery of a certain amount of the tertiary medication for a prolonged period of time to a targeted diseased area within the body.

In accordance with an embodiment of the present invention, the tertiary medication may be released in other parts of the body depending on the targeted diseased area within the body.

In accordance with an embodiment of the present invention, the tertiary medication comprises a drug selected from the group consisting of, but not limited to, azithromycin, haloperidol, co-amoxiclav, metformin, rosuvastatin, levofloxacin, moxifloxacin, erythromycin, clarithromycin, and ofloxacin.

In accordance with an embodiment of the present invention, a plurality of film coatings is adapted to cover the second middle layer (204) and the third inner layer (206), e.g. one film coating per layer. The plurality of film coatings prevent the second middle layer (204) and the third inner layer (206) from dissolving until the second middle layer (204) and the third inner layer (206) reach the lower gastrointestinal tract within the user's body, thereby releasing the secondary medication (210) and the tertiary medication into the bloodstream after a period of delay after oral ingestion or oral administration.

In accordance with an embodiment of the present invention, the plurality of film coatings are enteric coatings made up of acid-resistant polymers. Further, each of the film coating is made up of a material selected from the group consisting of, but not limited to, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate, trimellitate, sodium alginate, zein or enteric coating aqueous solution (ethylcellulose, medium chain triglycerides, oleic acid, sodium alginate, stearic acid).

In accordance with an embodiment of the present invention, each of the film coatings is an enteric coating made up of acid-resistant polymers and/or pH-sensitive polymeric coats to delay the release of the secondary medication (210) and tertiary medication into the bloodstream within the user's body. The pH-sensitive polymeric coats are configured to protect the second layer (204) and the third layer (206) of the composition (200) from the acidic environment within the user's stomach. Further, the acid-resistant polymers include, as disclosed supra.

In accordance with an exemplary embodiment of the present invention, the pharmaceutical composition (200) is designed using three different medications with multi-layer coatings and also applied in various dosage forms.

### Dosing

It is noted that the different layers (primary, secondary, tertiary) have different rates of absorption, and/or different targeted drug delivery areas with a patient's GI system. One of skill in the art would readily know of which layer(s) would comprise the main drug for treating a specific disease/disorder, and which layer(s) would comprise the other amelioration formulations for treating the adverse side effects for the main drug(s). The amelioration formulation(s) may have a different target area and different rate of absorption into patient's bloodstream than the main drug. The layer(s) that the amelioration formulations are placed within the pill is based on one or more factors that a skilled artisan could readily determine based on the pharmacokinetic properties of each layer, such as by way of non-limiting examples: rate of absorption of the main drug versus the amelioration formulas/layer(s); time when adverse side effects of main drug are experienced versus when the amelioration layers take effect; etc. the therapeutic time (e.g. activation and duration) deliver amelioration formula into the bloodstream before or after the main drug is delivered. (See also Table A).

When designing the pill layers, one of skill in the art will need to consider the pharmacokinetic parameters or profiles of each layer and the pill as a whole. As used herein, the term “pharmacokinetic parameters” refers to the characteristics of each layer comprising the main drug(s) or the amelioration of side effects ingredients. By way of non-limiting examples, pharmacokinetic parameters that one of skill in the art would use to design the amount, formulation, layer location, etc. of the pill herein, comprise one or more of factors listed in Table A that are well known to one in the art of drug pharmacokinetics (e.g. see—H M Jones, K Rowland-Yeo, “Basic Concepts in Physiologically Based Pharmacokinetic Modeling in Drug Discovery and Development”, CPT: Phannacometrics & Systems Pharmacology, August 2013: 2 (8); pages 1-12; the entirety of which are hereby incorporated in its entirety).

TABLE A
Severity of adverse side effects of main drug
Disease or disorder being treated
Location of target tissue
Severity or stage of disease or disorder
Rate of absorption of the main drug versus the
amelioration formulas/layer(s);
Time when adverse side effects of main drug are
experienced versus when the amelioration
layers take effect;
Cₘₐₓ, maximum observed concentration each layer;
Tₘₐₓ, time of achieved maximum observed concentration each layer;
AUC₀₋ₕ, is the estimated drug/ingredient area under the curve

It is noted that although the time of release of a layer of the pill is controlled in part by the formulation of each layer, the time that the layer actually takes effect may not be equivalent. For example, although layer one (outer) may release before layer two (inner), layer two may still have a faster dissolve rate and time to take physiological effect than layer one. One of skill in the art can readily determine which layers should comprise the main drug(s) and which layers should comprise the amelioration ingredients based on the pharmacokinetic profiles of specific the main drug(s) versus the specific amelioration ingredients and the specific side effects and their severity.

In an exemplary embodiment, the amelioration formulation/layer(s) are delivered before the main drug is released in order to further counter the main drug's adverse side effects. For example, the amelioration layers may coat the GI tract to protect it before release of a main drug that is known to irritate the GI lining.

In another exemplary embodiment, the main drug is released first, and then the amelioration formulations/layer(s) are released based on a pharmacokinetic computation of the amount of time when the main drug starts to actually take effect and generate adverse side effects, versus how long it takes for the amelioration layers to take effect to counter the adverse side effects.

The amount of amelioration formulas within the layers may also be different based upon how severe the adverse side effects are. For example, a patient who experiences severe nausea and headaches from taking a main drug would take a pill herein comprising an amelioration formula of about 25 mg. And a patient experiencing minor nausea and headaches would take a pill herein comprising an amelioration formula of about 10 mg.

### Method of Making

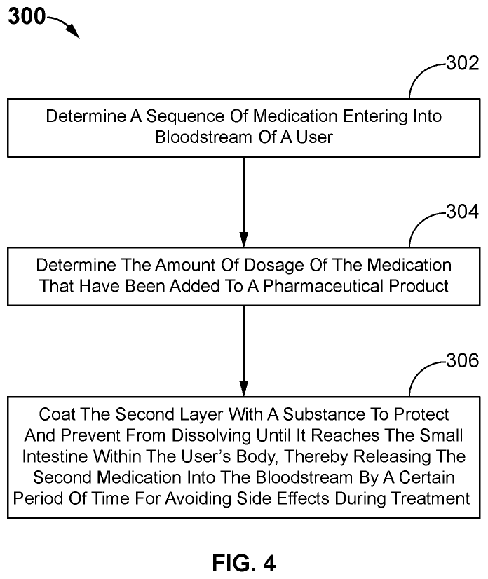

In accordance with an embodiment of the present invention, a method for preparing or designing the pharmaceutical composition of FIGS. 1 and 2 is disclosed. The method comprises different steps which includes, as disclosed in FIG. 4, step 302 for determining the sequence of medication release based on the preference of the medication entering into the user's bloodstream. Further, the method involves step 304 for determining the amount of dosage of the medication that has been added to the composition. The medications are compacted from a chemical powder into a solid dose to make the composition with one or more layers such as, the first layer (102) and the second layer (104). The method further involves step 306 comprising coating the second layer (104) with a substance to protect and prevent the second layer (104) from dissolving until the second layer (104) reaches the lower gastrointestinal tract within the user's body, thereby releasing the secondary medication within the second layer (104) into the user's bloodstream after a period of delay after oral ingestion for avoiding side effects during treatment.

In accordance with an embodiment of the present invention, the pharmaceutical composition (100) and (200) are designed with two or three separate tablets, respectively, with drugs and will be combined by using tablet in a tablet technology well known in the art, such as by way of non-limiting examples: Single station tablet press; Rotary tablet press; One or multi-layer rotary tablet press; Wurster column; Spray granulation; Wet granulation; Roller Compaction; Dry granulation; Hot Melt Extrusion; and Powder mixing. Exemplary manufacturers of tablet pressing, comprises by way of non-limiting examples: (e.g. manufactures—Fette™, Kilian™, Kikusui™, Bosch-Manesty™, DOT-Bonapace™, Natoli Eng.™, GEA™, and IMA™). Exemplary manufacturers of tablet pressing, comprises by way of non-limiting examples: Bosch™; Zanasi™; IMA™; MG2™; and DOT Bonapace™. Exemplary manufacturers of coating comprise by way of non-limiting examples: O'Hara™; Manesty™, Thomas Eng.™, IMA™, and Glatt™.

In accordance with an embodiment of the present invention, sugar spheres or granules in the pharmaceutical composition may be coated with one or more layers with different type of active drugs, sugar spheres or granules may be filled into a specific gelatin, HPMC or any other capsule type. Further, the sugar spheres or granules may be coated With any drug and then can be mixed in a specific ratio as required, followed by filling into a specific gelatin, Hydroxypropyl Methylcellulose (HPMC) or any other capsule type.

In accordance with an embodiment of the present invention, the pharmaceutical composition is designed with one or more layers of the drug or medication and may be filled into a specific gelatin, HPMC or any other capsule type.

For example, the method of preparing the pharmaceutical composition of FIG. 3 comprises: a first step of determining the sequence of medication release based on the preference of the medication (primary, secondary, tertiary); and a second step for determining the amount of dosage of each medication (primary, secondary, tertiary) that has been added to the composition. The method of preparing the pharmaceutical composition of FIG. 3 further comprises a third step of: coating the third inner layer (206) with a substance to protect and prevent the third inner layer (206) from dissolving until the third inner layer (206) reaches the lower gastrointestinal tract within the user's body, thereby releasing the tertiary medication within the third inner layer (206) into the user's bloodstream after a period of delay after oral ingestion for avoiding side effects during treatment.

In another or additional embodiment, the method of preparing the pharmaceutical composition of FIG. 3 further comprises a fourth step of: coating the second middle layer (204) with a substance to protect and prevent the second middle layer (204) from dissolving until the second middle layer (204) reaches a middle or lower gastrointestinal tract within the user's body, thereby releasing the secondary medication (210) within the second middle layer (204) into the user's bloodstream and/or inner intestinal lining after a period of delay after oral ingestion for avoiding side effects during treatment.

In accordance with an embodiment of the present invention, the substance coating could be an enteric coating. This coating is dissolved when the coating is in contact with a less acidic environment of the small intestine or with the digestive enzymes within the user's body.

In accordance with an embodiment of the present invention, the sequence of medication is determined by how quickly and how much of a drug reaches its intended target site of action. Further, the factors that affect absorption and therefore bioavailability include the way a drug is designed and manufactured, physical and chemical properties, ingredients, physiologic characteristics of the user, and how the medication is stored. For example, the pharmaceutical composition is designed to release the secondary medication (108) (of FIGS. 1 and 2) and the tertiary medication (of FIG. 3) into the user's bloodstream slowly or in repeated small amounts for a time period. This is called modified-release, controlled-release, sustained-release, or extended-release.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

1. A pharmaceutical composition (100), comprising:
a first outer layer (102) formed with a primary medication (106);
a second inner layer (104) formed with a secondary medication (108);
a film coating adapted to cover said second layer (104);
wherein said first layer (102) allows immediate release of said primary medication (106);
wherein said second layer (104) allows sustained release of said secondary medication (108);
wherein said film coating prevents said second layer (104) from dissolving until said second layer (104) reaches a lower gastrointestinal tract within a user's body, thereby releasing said secondary medication (108) into a bloodstream after a period of delay after oral ingestion; and
wherein said primary medication and said secondary medication are different medications with different pharmacokinetic parameters.

2. The pharmaceutical composition (100) according to claim 1, wherein:
said secondary inner medication (108) comprises one or more active agents or ingredients to treat or prevent a disease or disorder; and
said primary outer medication (106) comprises a plurality of additives able to reduce or prevent one or more adverse side effects associated with said secondary inner medication.

3. The pharmaceutical composition (100) according to claim 1, wherein:
said primary outer medication (106) comprises one or more active ingredients or agents to treat or prevent a disease or disorder; and
said secondary inner medication (108) comprises a plurality of additives able to reduce or prevent one or more adverse side effects associated with said primary outer medication.

4. The pharmaceutical composition (100) according to claim 1, wherein said secondary inner medication (108) comprises a drug selected from the group consisting of azithromycin, Haloperidol, Co-Amoxiclav, Metformin, Rosuvastatin, Levofloxacin, Moxifloxacin, Erythromycin, Clarithromycin, and Ofloxacin.

5. The pharmaceutical composition (100) according to claim 1, wherein said secondary medication (108) comprises azithromycin mixed with dibasic calcium phosphate anhydrous, pregelatinized starch, sodium croscarmellose, magnesium stearate, sodium lauryl sulfate, hypromellose, lactose, titanium dioxide or triacetin.

6. The pharmaceutical composition (100) according to claim 1, wherein said primary medication (106) comprises one or more of: domperidone, procyclidine, ranitidine, Co enzyme-Q10, vitamin D, and mebeverine.

7. The pharmaceutical composition (100) according to claim 1, wherein said primary medication (106) comprises domperidone mixed with lactose monohydrate or maize starch.

8. The pharmaceutical composition (100) according to claim 1, wherein said film coating is an enteric coating made up of one or more acid-resistant polymers.

9. The pharmaceutical composition (100) according to claim 1, wherein said film coating is made up of a material selected from the group consisting of methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate, trimellitate, sodium alginate, and zein or enteric coating aqueous solution (ethylcellulose, medium chain triglycerides, oleic acid, sodium alginate, stearic acid).

10. The pharmaceutical composition (100) according to claim 9, wherein said film coating is made up of cellulose acetate succinate.

11. The pharmaceutical composition (100) according to claim 1, wherein said composition is in the form of a tablet, a pill, or a capsule.

12. A pharmaceutical composition (200), comprising:
a first outer layer (202) formed with a primary medication (208);
a second middle layer (204) formed with a secondary medication (210);
a third inner layer (206) formed with a tertiary medication (212); and
a plurality of film coatings adapted to cover said second layer (204) and said third layer (206);
wherein said first outer layer (202) allows immediate release of said primary medication (208);
wherein said second middle layer (204) and said third inner layer (206) allows sustained release of said secondary medication (210) and said tertiary medication (212), respectively;
wherein said plurality of film coatings prevent said second middle layer (204) and said third inner layer (206) from dissolving until said second middle layer (204) and said third inner layer (206) reach a lower gastrointestinal tract within a user's body, thereby releasing said secondary medication (210) and said tertiary medication (212) into a bloodstream after a period of delay after oral ingestion, and said primary, secondary, and tertiary medications comprise different pharmacokinetic parameters; and
wherein said primary, secondary, and/or tertiary medications comprise at least one additive for preventing one or more adverse side effects from the pharmaceutical composition.

13. The pharmaceutical composition (200) according to claim 12, wherein:
said tertiary medication (212) comprises one or more active agents or ingredients for treating or preventing a disease or disorder; and
said primary medication (208) and said secondary medication (210) comprise a plurality of additives for preventing one or more adverse side effects associated with the tertiary medication.

14. The pharmaceutical composition (200) according to claim 12, wherein said tertiary medication (212) comprises a drug selected from the group consisting of azithromycin, haloperidol, Co-amoxiclav, metformin, rosuvastatin, levofloxacin, moxifloxacin, erythromycin, clarithromycin, and ofloxacin.

15. The pharmaceutical composition (200) according to claim 12, wherein each of said primary medication (208) and said secondary medication (210) comprise one or more of: domperidone, procyclidine, ranitidine, Co enzyme-Q10, vitamin D, and mebeverine.

16. The pharmaceutical composition (200) according to claim 12, wherein said plurality of film coatings are enteric coatings made up of acid-resistant polymers.

17. The pharmaceutical composition (200) according to claim 12, wherein each of said film coatings is made up of a material selected from the group consisting of methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate, trimellitate, sodium alginate, and zein or enteric coating aqueous solution.

18. The pharmaceutical composition (200) according to claim 12, wherein said composition is in the form of a tablet, a pill, or a capsule.

19. The pharmaceutical composition (200) according to claim 12, wherein:
said primary medication (208) comprises one or more active agents or ingredients for treating or preventing a disease or disorder; and
said secondary medication (210) and said tertiary medication (212) comprise a plurality of additives for preventing one or more adverse side effects associated with the primary medication.

20. The pharmaceutical composition (200) according to claim 12, wherein:
said primary medication (208) and said secondary medication (210) each comprise one or more active agents or ingredients for treating or preventing a disease or disorder; and
said tertiary medication (212) comprises a plurality of additives for preventing one or more adverse side effects associated with the primary medication and/or the secondary medication.

21. The pharmaceutical composition (200) according to claim 12, wherein:
said secondary medication (210) and said tertiary medication (212) each comprise one or more active agents or ingredients for treating or preventing a disease or disorder; and
said primary medication (208) comprises a plurality of additives for preventing one or more adverse side effects associated with the primary medication and/or the secondary medication.

22. The pharmaceutical composition (200) according to claim 12, wherein:
said secondary medication (210) comprises one or more active agents or ingredients for treating or preventing a disease or disorder; and
said primary medication (208) and said tertiary medication (212) comprise a plurality of additives for preventing one or more adverse side effects associated with the primary medication and/or the secondary medication.

23. The pharmaceutical composition (200) according to claim 12, wherein:
said primary medication (208) and said tertiary medication (212) each comprise one or more active agents or ingredients for treating or preventing a disease or disorder; and
said secondary medication (210) comprises a plurality of additives for preventing one or more adverse side effects associated with the primary medication and/or the secondary medication.

24. A method for preparing a pharmaceutical composition, comprising:
determining a sequence of a plurality of medications to be released based on a preference of sequence of each medication entering into the user's bloodstream or intestinal lining or stomach;
determining an amount of dosage of each medication to add to a layer of the pharmaceutical composition;
wherein the medications are compacted from a chemical powder into a solid dose to make the pharmaceutical composition with two or more layers, and the medications comprise different pharmacokinetic parameters.

25. The method for preparing a pharmaceutical composition of claim 24, wherein the pharmaceutical composition comprises:
a first outer layer (102) formed with a primary medication (106);
a second inner layer (104) formed with a secondary medication (108); and
coating the second inner layer (104) with a substance to protect and prevent the second inner layer (104) from dissolving until the second inner layer (104) reaches the lower gastrointestinal tract within the user's body, thereby releasing the secondary medication within the second inner layer (104) into the user's bloodstream after a period of delay after oral ingestion for avoiding side effects during treatment.

26. The method for preparing a pharmaceutical composition of claim 24, wherein the pharmaceutical composition comprises:
a first outer layer (202) formed with a primary medication (208);
a second middle layer (204) formed with a secondary medication (210);
a third inner layer (206) formed with a tertiary medication; and
coating the second middle layer (204) with a first substance to protect and prevent the second middle layer (204) from dissolving until the second middle layer (204) reaches a stomach, or a middle or lower gastrointestinal tract within the user's body; and
coating the third inner layer (206) with a second substance to protect and prevent the third inner layer (206) from dissolving until the third inner layer (206) reaches the lower gastrointestinal tract within the user's body, thereby releasing the tertiary medication within the third inner layer (206) into the user's bloodstream after a period of delay after oral ingestion for avoiding side effects during treatment.
